(19) United States
(12) Patent Application Publication
(10) Pub. No.: US 2012/0266873 A1
(43) Pub. Date: Oct. 25, 2012

(54) INTEGRATED POSITIVE AIRWAY PRESSURE APPARATUS

(75) Inventor: Michael Gerard Lalonde, Alpharetta, GA (US)

(73) Assignee: DESHUM MEDICAL, LLC., Cambridge, MA (US)

(21) Appl. No.: 13/503,274

(22) PCT Filed: Oct. 20, 2010
(86) PCT No.: PCT/US10/53370

§ 371 Date: Jul. 13, 2012

Related U.S. Application Data
(60) Provisional application No. 61/253,500, filed on Oct. 20, 2009.
(60) Provisional application No. 61/288,290, filed on Dec. 19, 2009.
(60) Provisional application No. 61/301,151, filed on Feb. 3, 2010.

(51) Int. Cl. A61M 16/06; A61M 16/00; A61M 16/20

(52) U.S. Cl. 128/201.13; 128/205.24; 128/205.25

(57) ABSTRACT

A gas delivery system that provides positive airway pressure therapy. A mask couples to a patient's face to deliver pressurized gas to an airway of the patient. The mask includes a flow generator system disposed on the mask and that pressurizes the gas, the flow generator including at least one motor. A controller controls the at least one motor.

### CROSS-REFERENCE TO RELATED APPLICATIONS

This application claims the benefit of priority under 35 U.S.C.§119(e) of U.S. provisional application Nos. 61/288,290, filed on Dec. 19, 2009; 61/253,500, filed on Oct. 20, 2009; and 61/301,151, filed on Feb. 3, 2010. The entire contents of provisional application Nos. 61/288,290, 61/253,500 and 61/301,151 are incorporated herein by reference.

### BACKGROUND

1. Field of the Disclosure

The present disclosure relates to a gas delivery system. One example of the gas delivery system provides positive airway pressure therapy during a patient's sleep period.

2. Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Certain individuals have difficulty breathing during sleeping due to a collapse or obstruction of airways. For example, obstructive sleep apnea (OSA) may occur when the body relaxes during sleep, and the upper airway of the sleeping individual collapses, either partially or completely, to obstruct breathing during sleep. This condition is particularly common in overweight individuals, individuals with large necks, or individuals who abuse alcohol.

One treatment for the above-noted condition is the application of a continuous positive airway pressure apparatus (CPAP). The CPAP apparatus typically comprises a base unit placed near the patient's bed connected to a mask unit via a flexible hose. Due to difficulties caused by connection to the base unit via the flexible hose, compliance with treatment via a CPAP unit is often less than optimum. For example, the patient's movement is restricted by the hose. Additionally, back pressure or sensory lag time in response to changes in conditions may be caused by the hose. Moreover, the base unit may require a larger blower unit in order to overcome the pressure drop between the base unit and the mask unit. The large blower unit, in some cases, produces an undesirable level of noise.

Accordingly, the present disclosure and embodiments recited in the attached claims may ameliorate one or more of the above-noted difficulties with conventional therapies for OSA.

### SUMMARY

The foregoing paragraphs have been provided by way of general introduction, and are not intended to limit the scope of the following claims. The described embodiments, together with further advantages, will be best understood by reference to the following detailed description taken in conjunction with the accompanying drawings.

One aspect of the invention includes a gas delivery system that provides positive airway pressure therapy during a patient's sleep period. In this aspect, the system includes a mask that couples to a patient's face to deliver pressurized gas to an airway of the patient. The system further includes a flow generator system that directly detachably couples to the mask and pressurizes the gas.

Another aspect of the gas delivery system includes a mask that couples to a patient's face to deliver pressurized gas to an airway of the patient. The mask typically includes a flow generator system that pressurizes the gas. The flow generator system includes at least one brushless motor.

One aspect of the gas delivery system includes a mask that couples to a patient's face to deliver pressurized gas to an airway of the patient. The mask typically includes a flow generator system that pressurizes the gas. The flow generator system includes at least one motor. An acoustic damper unit is disposed upstream of the flow generator system.

Another aspect of the invention includes a mask that couples to a patient's face to deliver pressurized gas to an airway of the patient. The mask typically includes a flow generator system that pressurizes gas. The flow generator system includes at least one motor. At least one washout vent allows fluid communication, separately from the flow generator system, between an exterior of the mask and an interior of the mask. A check-valve obstructs the at least one wash out vent during inspiration by the patient and allows gas flow through the at least one wash out vent during expiration by the patient.

One aspect of the invention includes a gas delivery system that provides positive airway pressure therapy. A mask couples to a patient's face to deliver pressurized gas to an airway of the patient. The mask includes a flow generator system disposed on the mask and that pressurizes the gas, the flow generator includes at least one motor. A controller controls the at least one motor according to a power management system.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a process and instrumentation diagram and schematic representation of a background CPAP apparatus;

FIG. 2 is a perspective view of one embodiment of an integrated positive airway pressure (PAP) apparatus;

FIG. 3 is a left side view of the PAP apparatus depicted in FIG. 2;

FIG. 4 is an exploded view of the PAP apparatus depicted in FIG. 2;

FIG. 5A is a perspective view of a power unit that may be used to supply power to the PAP apparatus of FIG. 2;

FIG. 5B is a detailed view of the connectors used with the power unit depicted in FIG. 5A;

FIG. 6A is a front view of a flow generator that may be used with the PAP unit of FIG. 2;

FIG. 6B is a section view taken along lines A-A of the flow generator depicted in FIG. 6A;

FIG. 6C is a section view taken along lines A-A of the flow generator depicted in FIG. 6A, but with the inclusion of a gear system connected to the motor used to drive the flow generator depicted in FIG. 6A;

FIG. 7A is a schematic representation of a gas flow path of the PAP unit depicted in FIG. 2;

FIG. 7B is a section view taken along lines A-A in FIG. 2A;

FIG. 8 is a detailed view of an acoustic damper typically used in conjunction with the PAP unit depicted in FIG. 2;

FIG. 9 is a perspective view of an embodiment of a PAP unit in which a flow generator is detachably couplable to a mask portion (patient interface);

FIGS. 10A and 10B depict the detachable flow generator coupled to a base unit, which is in turn connected to a hose for connection to an alternate mask unit;

FIG. 11 is a general system schematic for a PAP unit as described in FIGS. 2-10, and 12; and

FIG. 12 is a flow chart depicting one embodiment of power management for the various PAP unit embodiments described herein.

### DETAILED DESCRIPTION OF THE EMBODIMENTS

Referring now to the drawings, wherein like reference numerals designate identical or corresponding parts throughout the several views.

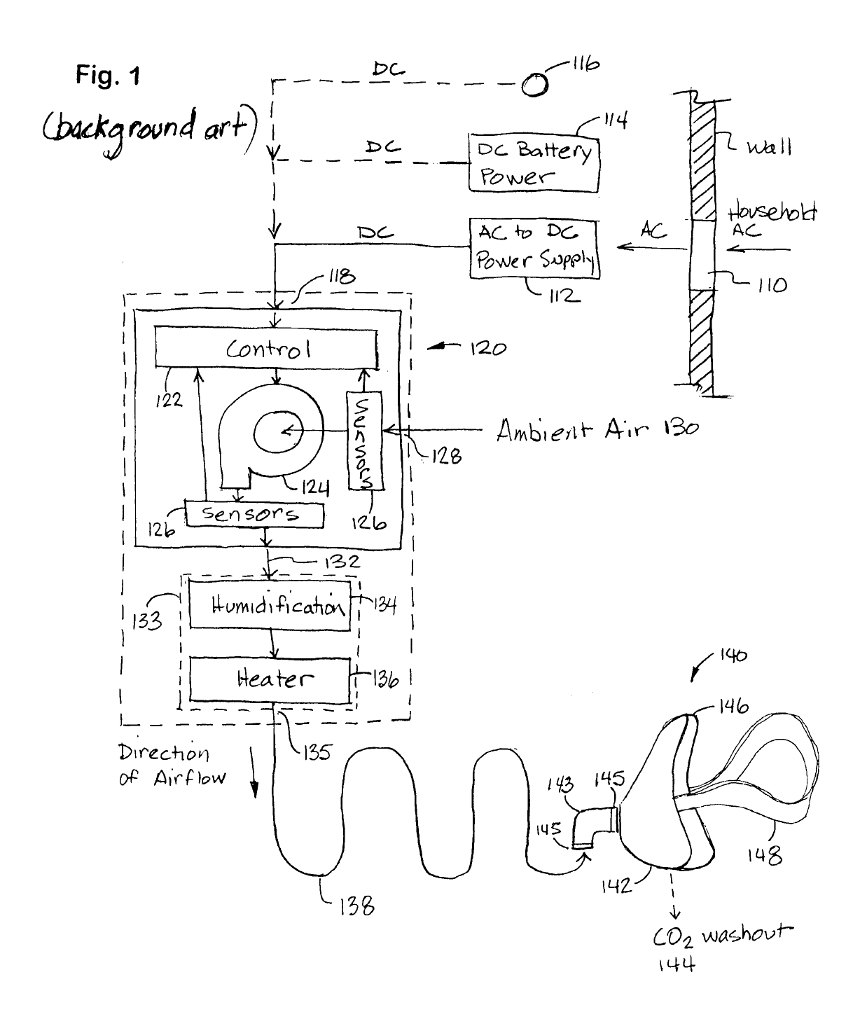

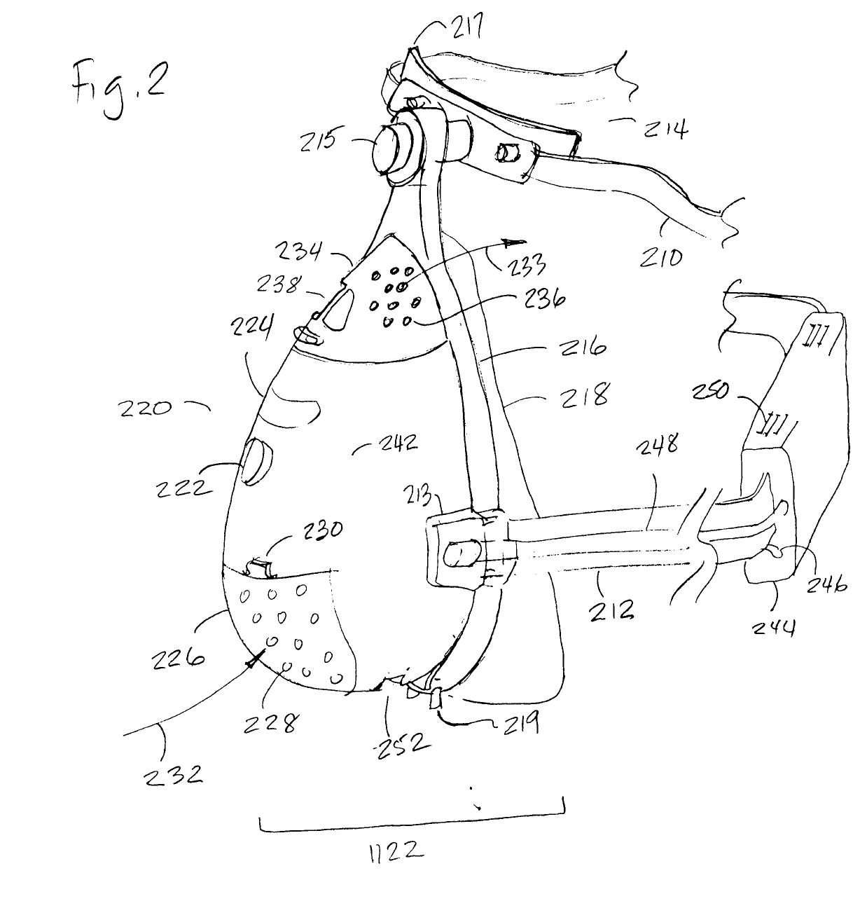

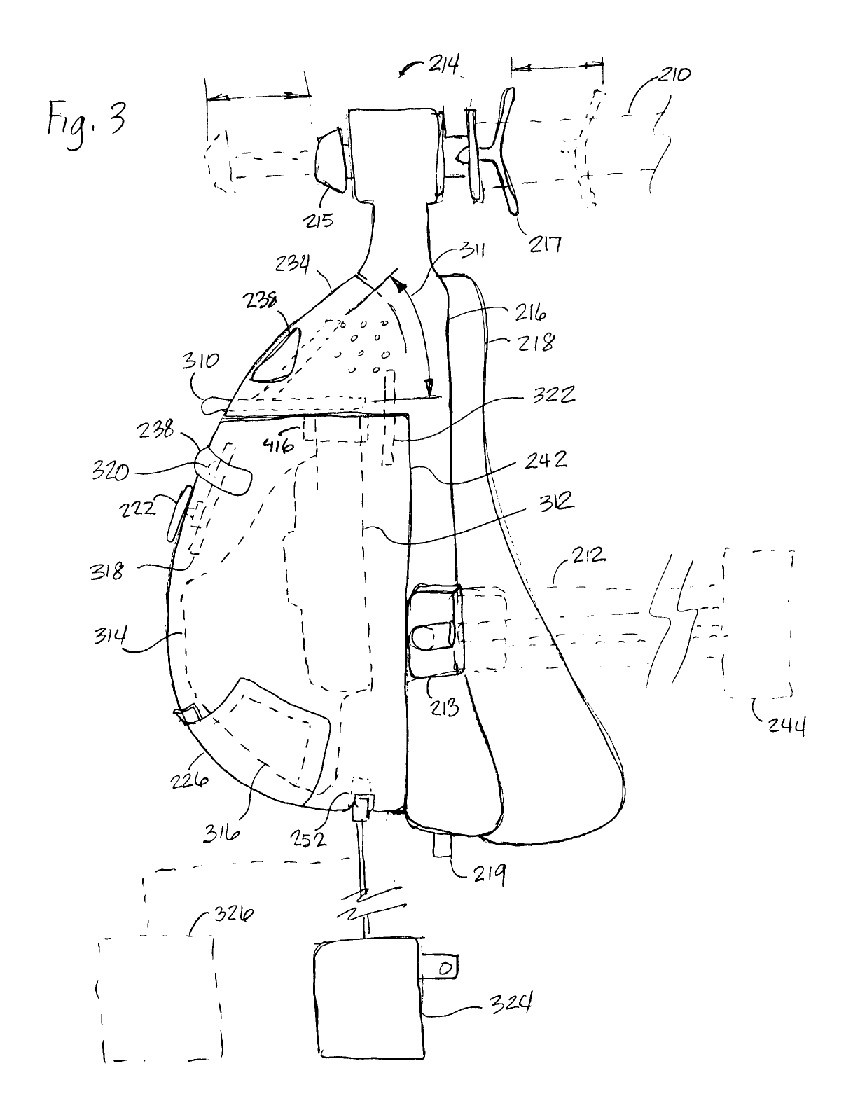

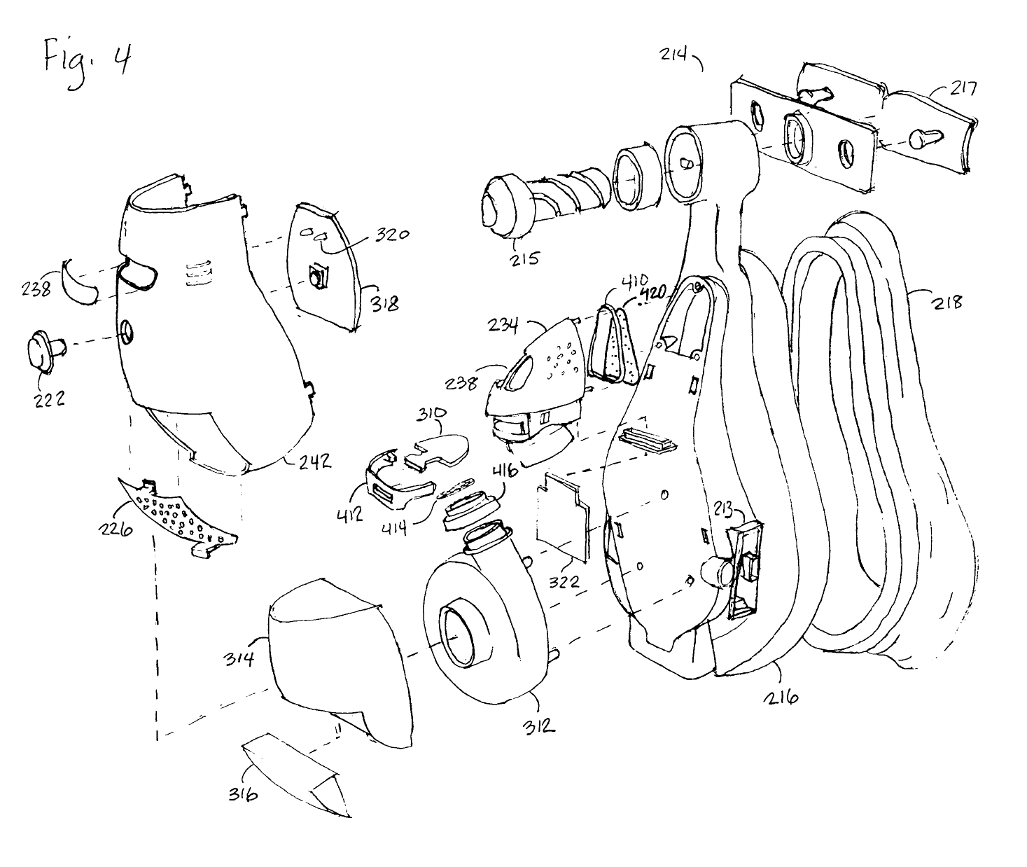

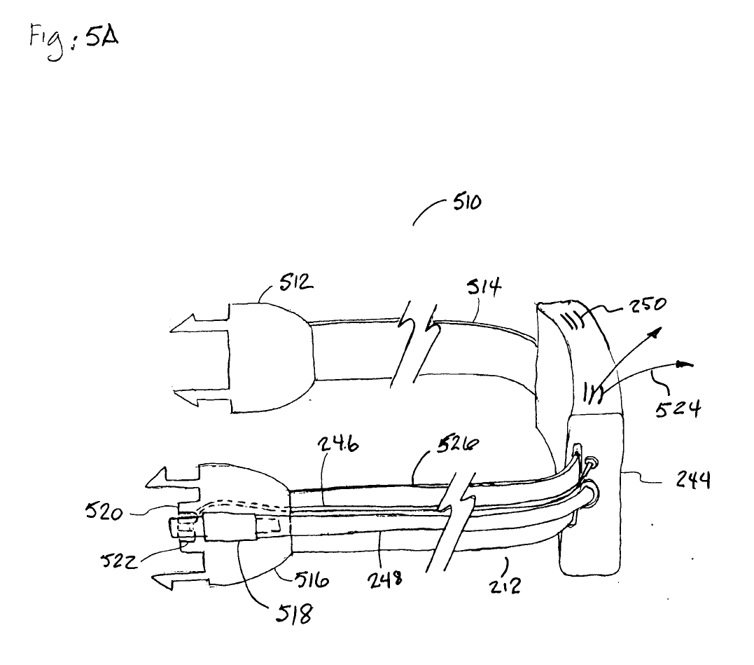

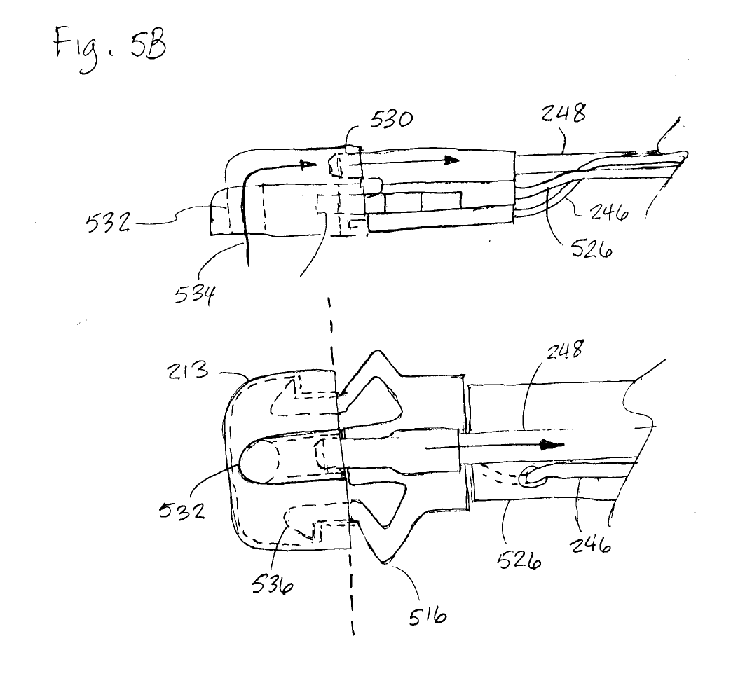

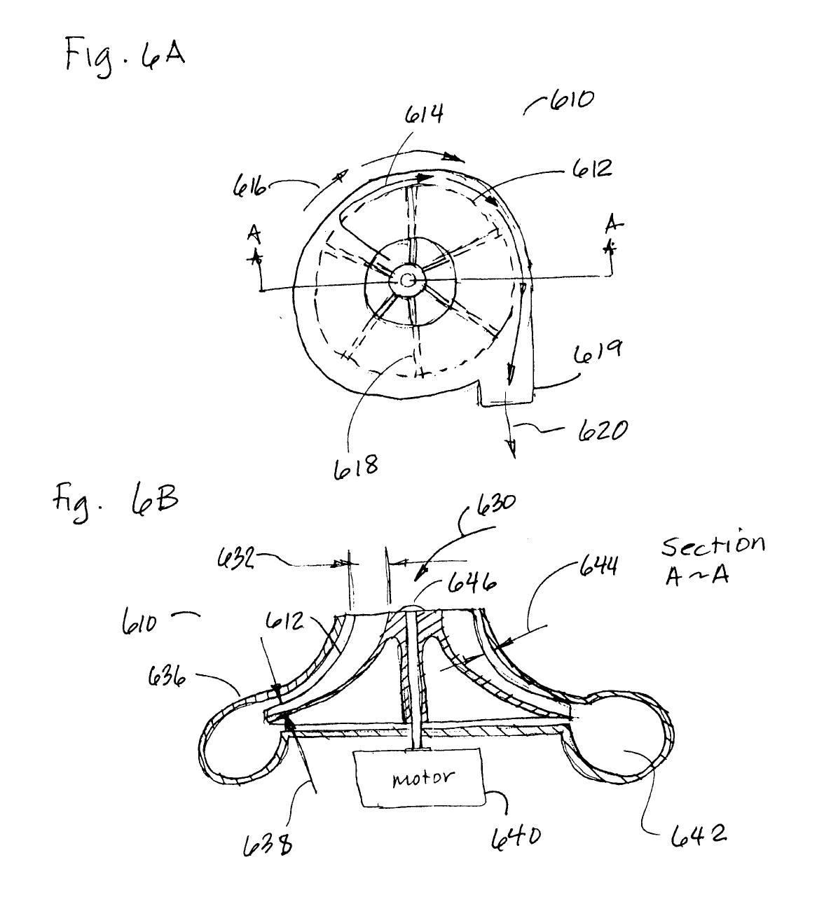

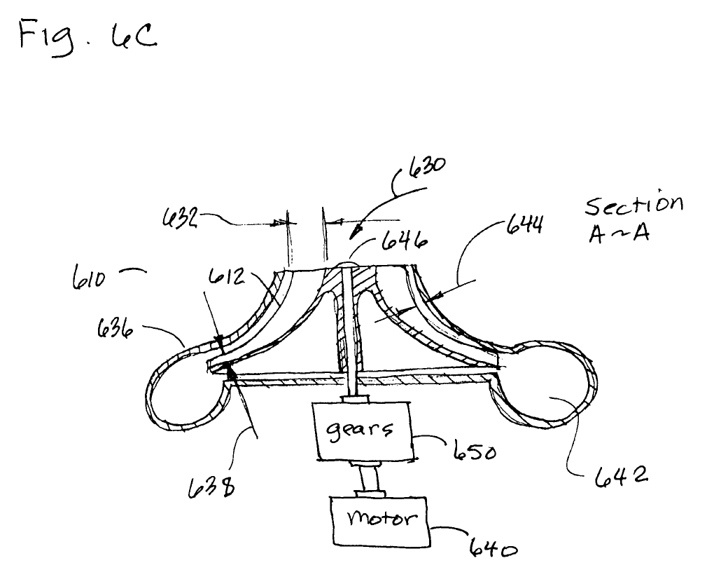

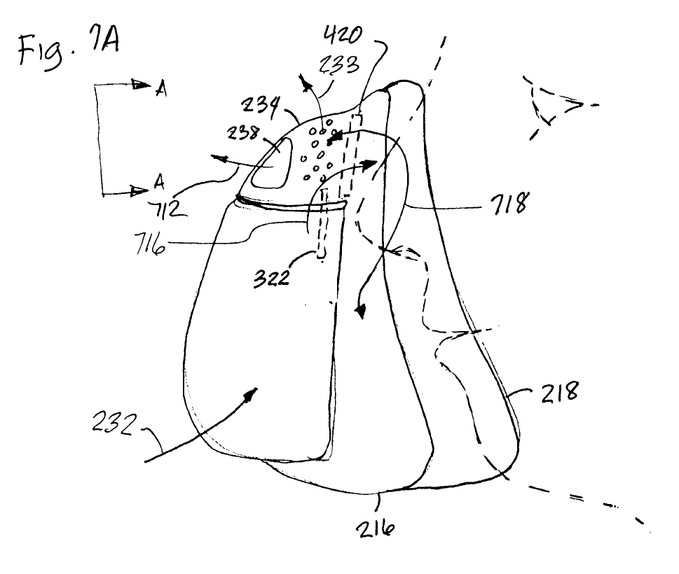

FIG. 1 depicts a background CPAP device in which a flow generator 120 includes a compressor 124 controlled by an electronic controller 122 and which monitors the flow rate and pressure of ambient air 130 drawn into the compressor 124 via sensors 126. The ambient air, as shown in FIG. 1, is drawn directly into the flow generator 120 via an air inlet 128.

In the most common embodiments, the flow generator 120 is operated on alternating current (AC) power using an AC to direct current (DC) power supply 112. AC, supplied via a typical household outlet 110 is converted in the power supply 112 to DC power that ultimately drives the flow generator 120. Occasionally, the power supply is incorporated within flow generator enclosure 120. Also, the DC power used to drive the flow generator 120 may be supplied via another supply, for example, a cigarette lighter-type DC power port 116 on an automobile or via a DC battery pack 114 as shown in FIG. 1.

The flow generator 120 may also include a humidifier 134 connected to an outlet of the compressor 124, for example, via a connection 132 as shown in FIG. 1. The humidifier is typically connected to a reservoir (not shown) to supply moisture to the pressurized gas ultimately supplied to the patient. In some cases, the amount of moisture supplied to the patient may be in the range of 700 ml per sleep period.

The conventional CPAP unit shown in FIG. 1 may also include a heater 136. The humidifier 134 in combination with the heater 136 comprises a conditioning unit 133. Eventually, via a connector such as connector 135, a hose 138 typically connects to a connector 145 on a mask 140. In some cases, the connector may include an elbow 143, and the connector may itself swivel. The mask 140 typically includes a hole for expiration of the patient, and the hole is commonly referred to as a “washout vent.” In FIG. 1, the washout vent 144 allows gas expelled during expiration to leave the mask 140. Typically, the washout vent 144 is merely one or more open holes placing the interior of the mask in fluid communication with an exterior of the mask.

The mask typically includes a cushion surface 146 connected to a more rigid shell 142, and the mask is attached to the patient's head via flexible straps 148.

As discussed previously, certain complications are involved when using a hose such as hose 138. Accordingly, it is beneficial to reduce reliance on a connection such as hose 138 in the PAP unit disclosed herein.

FIG. 2 describes an integrated PAP unit 1122. The integrated PAP unit 1122 depicted in FIG. 2 includes a flow generator 220 which is either connected to or encased in a rigid mask shell 216 and covered with a flow generator cap 242. The rigid mask shell 216 and/or flow generator cap 242 are typically formed from a plastic or light-weight metal. For example, these components may comprise polystyrene, polycarbonate, polyvinyl carbonate, polypropylene, etc. In one embodiment, one or more of the above-noted components is transparent.

In the embodiment depicted in FIG. 2, a power supply enclosure 244, which may include batteries, is connected via a strap 212 to the integrated PAP unit 1122. The strap 212 may be adjustable such that the power supply 244 may be supported at the back of a patient's neck. While a preferred location is on the back of the neck, other locations, such as the arm, shoulder, hip, or chest etc. may be used. In one embodiment, a cooling supply conduit 248 supplies gas from the integrated PAP unit 1122 to the power supply 244. One benefit of this arrangement is that, for example, when the power supply 244 includes batteries or a fuel cell for power, heat generated when the integrated PAP unit 1122 is in use may be dissipated to preserve the life of the power supply 244 and to increase the comfort of the patient. To increase the effectiveness of supplied cooling, the power supply 244 may include vents 250 as shown in FIG. 2. An electrical conductor 246 typically follows along the strap 212 and ultimately connects to the integrated PAP unit 1122 to supply electrical power to the integrated PAP unit 1122. The strap 212 may be detachable from the integrated PAP unit 1122, for example, via a strap connector 213 shown in FIG. 2 and in more detailed description of FIG. 5A.

The integrated PAP unit 1122 may be supported further by a head rest assembly 214 shown in the upper part of FIG. 2. The head rest assembly typically includes a head rest adjustor 215 that may be pivotable and/or threadable so as to increase or decrease the pressure on the patient's head so that the integrated PAP unit 1122 firmly fits across the patient's nasal area, mouth area, or both. The head rest assembly may be secured to the patient's head via an upper strap 210. Typically, the upper strap 210 is adjustable and/or elastic to adjust to the circumference of the patient's head. Furthermore, the head rest assembly 214 may include a head rest cushion 217 as shown in FIG. 2 to increase the comfort of the patient. In another embodiment, head rest may also include certain sensors which contact the forehead in headrest cushion 217 such as those sensors employed in polysomnogram for measuring brain activity and/or sensors for motion, acceleration, skin perspiration, humidity, nerve electrical activity, which sensor activity is then commentated to controller 1168 of flow generator 220.

The area of the integrated PAP unit 1122 below the head rest assembly 214 is configured to couple to the patient's face. In this regard, a patient interface cushion 218 typically comprises a compliant material. In one example, the compliant material includes silicone, gel, foam, or another such compliant material and is configured to form a relatively gas-tight interface between the remainder of the integrated PAP unit 1122 and the patient's face. In still another embodiment, this cushion may also include such as those sensors employed in polysomnogram for measuring brain activity and/or sensors for motion, acceleration, skin perspiration, humidity, nerve electrical activity, which sensor activity is then commentated to controller 1168 of flow generator 220.

The integrated PAP unit 1122 shown in FIG. 2 typically includes auxiliary gas ports 219 for introduction to the integrated PAP unit 1122 of auxiliary gases such as O₂, medicinal substances, and/or moisture. Additionally, or alternatively, the auxiliary gas ports 219 may be used to receive a sensor used to collect data regarding the gas within the integrated PAP unit 1122. For example, the data may include one or more of an oxygen level, CO₂ level, pressure, acoustic vibrations, nitrogen levels, methyl nitrate levels, gas flow velocity, gas volume displacement, temperature, relative position of the integrated PAP unit 1122, motion, acceleration, skin perspiration, humidity, nerve electrical activity, infrared signals sent to or from the integrated PAP unit 1122, and other such information. Although the auxiliary gas ports 219 shown in FIG. 2 are disposed at the bottom of the integrated PAP unit 1122, other locations on the integrated PAP unit 1122 may be used, for example, a side position. Adjacent the auxiliary gas ports 219 in FIG. 2 is a power/data connection receptacle 252, which may be used to receive a cable from a remote control 1014 and/or a computer and/or alternate power supply. For example, during or after use of the integrated PAP unit 1122, data logged by a controller disposed on the integrated PAP unit 1122 may be downloaded for analysis by a physician. This data may relate to the above-noted parameters measured via sensors connected in the auxiliary gas ports 219 or sensors disposed in another area of the integrated PAP unit 1122. Thus, parameter levels with respect to time may be recorded in the integrated PAP unit 1122 and plotted or otherwise analyzed outside the integrated PAP unit 1122. In one embodiment, the air flow and/or pressure verses time relationship may be plotted, and it may be determined whether the integrated PAP unit has remedied the sleeping difficulties of the patient.

As the integrated PAP unit depicted in FIG. 2 may include a controller, it is often beneficial to provide a control button 222 for easy access by the patient wearing the integrated PAP unit 1122. In one embodiment, the control button 222 is disposed on the upper area, typically aligned with the nasal area of the patient. However, other areas may be convenient, depending on the preferences of the user, and the location of the control button 222 is not limited to the upper part of the integrated PAP unit 1122.

As further shown in FIG. 2, the integrated PAP unit 1122 may include an IR (infrared) transmit/receive device. This device may be used to transmit the above-noted data. Additionally, or alternatively, the IR device 224 may be used to control the operation of the integrated PAP unit 1122, for example, via a remote control similar to a remote control used for operation of a television. One benefit of this arrangement is that the patient may conveniently control the integrated PAP unit 1122, even while the integrated PAP unit 1122 is worn on the patient's face and also potentially when patient is in a prone position.

As further shown in FIG. 2, ambient air will travel through a flow generator intake flow path 232 past orifices such as flow generator intake holes 228. The flow generator intake holes 228 may be, in turn, disposed on a flow generator intake door such as flow generator door 226. One benefit of this arrangement is that the flow generator intake holes may be changed in size by replacement of the flow generator intake door 226. Another benefit of this arrangement is that the flow generator intake door can be opened to replace intake filter 316 occasionally. In one example, the flow generator intake door 226 is connected to the remainder of the integrated PAP unit 1122 via a flow generator intake door latch 230.

The integrated PAP unit 1122 further includes a gas flow diverter 234, which is used to divert expiration gas flow from the patient from within the integrated PAP unit 1122 to an exterior of the unit via unassisted breathing vent 238. Additionally, the integrated PAP unit 1122 typically includes washout vents 236 which are continually operable. In other words, the washout vents 236 remain open during normal operation of the integrated PAP unit 1122. In one embodiment, the unassisted breathing vents 238 are regulated via a flap or check valve to be described later. The check valve covers the unassisted breathing vents during pressurization by the compressor and inspiration by the patient and opens the unassisted breathing vents 238 during expiration by the patient. Gas pressure generated by the flow generator, in combination with the inspiration and expiration by the patient, causes the check valve to change state. In other words, when the check valve is in a relaxed position, the check valve covers the flow generator's outlet. However, when the flow generator is in operation, the pressure generated by the flow generator pushes the check valve against the unassisted breathing vents 238, thus closing the vents and allowing the patient to inspire gas passing primarily through the flow generator without also drawing a sizable volume of air from outside of the integrated PAP unit 1122. Next, when the patient expires, the increase in pressure within the integrated PAP unit 1122 overcomes the pressure generated by the flow generator and allows expired gases to escape through the washout vents to outside of the integrated PAP unit 1122. At all times while pressure is created by the flow generator, the check value flap is force open. Therefore, expired gas is less likely or is substantially prevented from passing backwards through the flow generator. When the flow generator does not provides sufficient pressure to force the check value flap open, unassisted breathing vents 238 are uncovered and the patient can freely breath through these same ports.

The unassisted breathing vent 238 allows direct communication between the interior of the integrated PAP unit 1122 and the exterior of the integrated PAP unit 1122 when flap is in close to flow generator.

FIG. 3 depicts a side view of the integrated PAP unit 1122 shown in FIG. 2. Certain reference numbers from FIG. 2 are repeated in FIG. 3 and will not be further discussed unless necessary. As shown in FIG. 3, the power and data connector receptacle 252 may be connected to an AC adaptor 324 or automobile circuit adaptor 326 to provide power in place of the power supply 244 or battery power supply 1210. One benefit of this arrangement is that a practically unlimited supply of power may be available by replacing the power supply 244, and even though the user is somewhat tethered to the AC adaptor 324 or automobile circuit adaptor 326, the connection between these components and the power and data connector receptacle 252 is relatively thin in comparison to the hose used in typical CPAP apparatuses. Therefore, the patient typically has a greater sense of freedom when using the integrated PAP unit 1122 in comparison to conventional CPAP units.

As discussed previously, the integrated PAP unit 1122 may include a check valve controlling flow between an interior and exterior of the unit via the unassisted breathing vents 238. In one embodiment, the check valve includes a diverter flap 310 that travels along a path 311 to open and close the outlet of a compressor 416. In the upper position along the path 311, the diverter flap 310 closes the unassisted breathing vents 238.

As shown by the hidden lines in FIG. 3, the integrated PAP unit 1122 typically includes a compressor 312, which generates the increase in pressure between the ambient air and the gas supplied to the patient. In one embodiment, the compressor 312 is driven by a brushless motor. In another embodiment, the compressor 312 is driven by a motor including a commutator. In certain embodiments, the motor and/or controller driving the compressor 312 are enclosed with a Faraday cage or other such electromagnetic emission limiting device.

In the embodiment depicted in FIG. 3, the compressor 312 draws ambient air through an intake filter 316 disposed inside of the flow generator intake door 226. The air then typically travels through some form of sound-abatement device such as an acoustic damper 314. In one embodiment, the acoustic damper is shown in FIG. 8. The compressor 312 increases the pressure of the air, which may or may not be combined with an additional gas via auxiliary gas ports 219, and discharges the pressurized gas at an outlet 416 for inspiration by the patient.

In the embodiment shown in FIG. 1, a sensor board 322 is disposed downstream of the compressor 312 and may be used to monitor any one of the parameters noted above, i.e., pressure, temperature, O₂ level, CO₂ level, etc.

An infrared transceiver 320 may communicate with external devices, such as a remote control by sending a signal through the IR transmit/receive lens 224. In another embodiment, a radio transceiver communicates to such as a remote control.

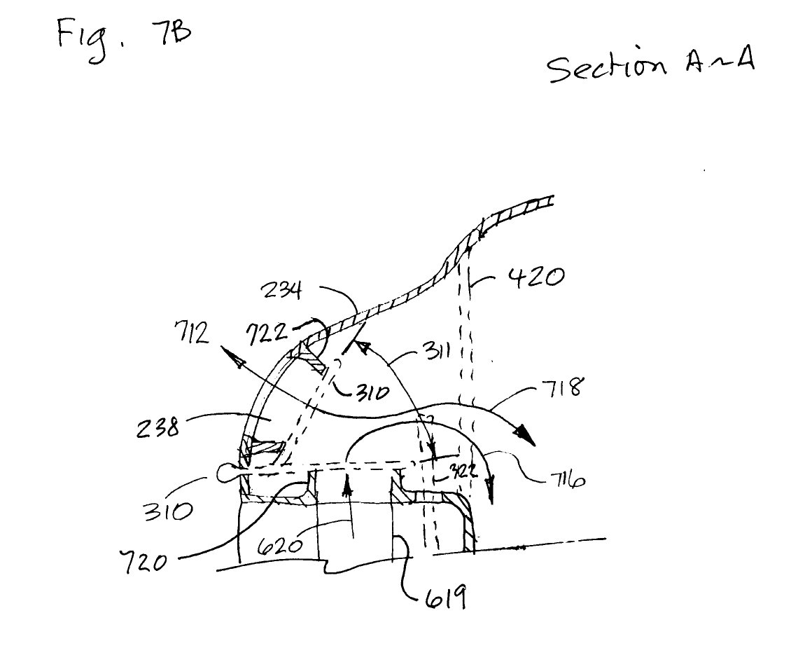

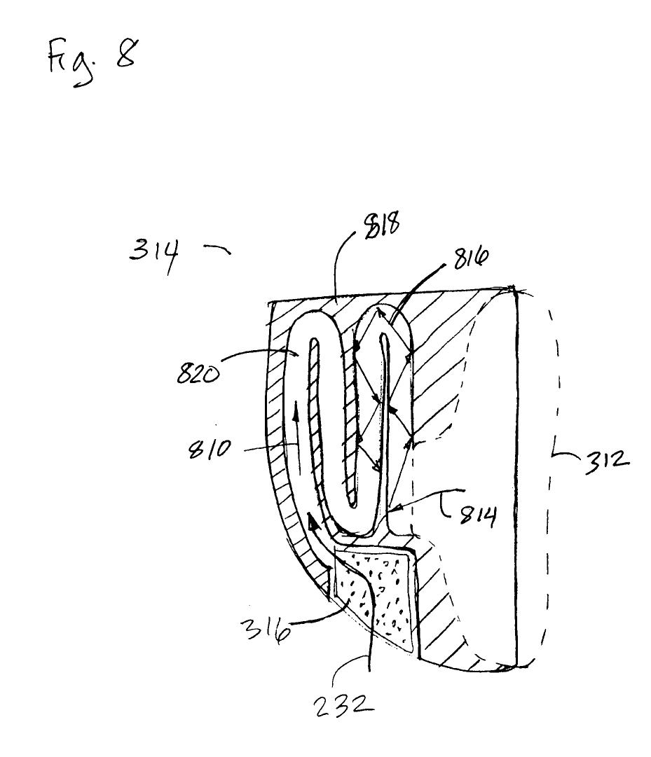

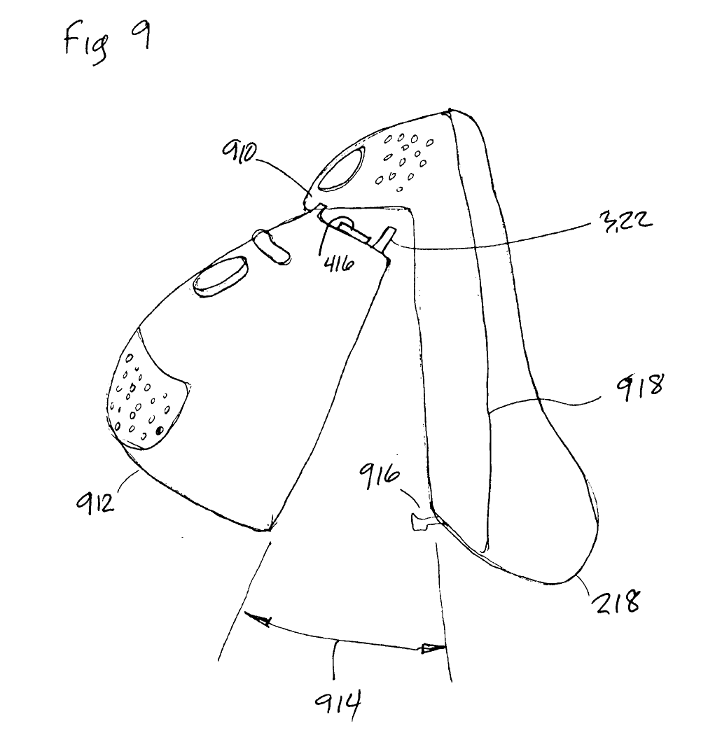

FIG. 4 is an exploded view of the integrated PAP unit 1122 depicted in FIG. 3 and FIG. 2. In the exploded view, it is evident that the gas flow diverter gasket 410 mates with a membrane and/or heat exchanger. In one embodiment, the membrane 420 may comprise a polyamide, polypropylene, or other gas-permeable membrane capable of inhibiting the passage of water vapor or liquid water. One benefit of the above-noted arrangement is that, as gas is expired by the patient, water vapor within the expired gas is obstructed by the membrane 420. In this regard, the moisture may be collected and used to further humidify the air discharged from the compressor 312. In this way, it is possible to reduce or eliminate the need for an external humidifier such as the one depicted in FIG. 1 for a conventional CPAP apparatus. As noted previously, a patient may require approximately 700 ml of water vapor to properly humidify the gas stream supplied to the mask with a conventional CPAP unit. In contrast, with the above-noted membrane 420, water vapor is actually recycled, at least to some extent, and the introduction of humidity via an external humidifier may be unnecessary.

As further shown in FIG. 4, the diverter flap, which is typically a flexible, substantially planar component, may be attached to a diverter flap retainer 412. As shown in FIG. 4, the diverter flap 310 may include a wide portion connected to a remainder of the diverter flap 310 via a thinner portion or neck. In this manner, the diverter flap may be detachably connected to the diverter flap retainer 412 and easily removed when worn, or when a different type of performance is desired.

An optional particulate screen 414 is shown in FIG. 4 as a discharge portion of the compressor 312. In some embodiments, the particulate screen 414 is connected to the outlet portion of the compressor 312 via a transition coupling 416.

As described with reference to FIG. 3, the integrated PAP unit 1122 may include a sensor board 322 for sensing various parameters in the gas discharged from the compressor 312. In order to allow electronic communication between the sensor board 322 and the data connector receptacle 252, the integrated PAP unit 1122 typically includes an interface 418 connectable to the sensor board 322. In this manner, should the sensor board 322 be damaged or expire due to chemical consumption, the sensor board 322 may be easily replaced.

As noted above, the membrane 420 may act as an obstruction to moisture expired by the patient. Additionally, the membrane 420 may be replaced or supplemented with a heat exchanger that absorbs heat from the gas expired by the patient. When the patient then inspires, the heat absorbed by the heat exchanger will be released into the gas stream as the patient inspires. Thus, in contrast to the conventional CPAP unit described in FIG. 1, some embodiments of the integrated PAP unit 1122 may not need an external heater.

As shown in FIG. 5A, the power supply 244 may be part of a battery and strap assembly 510 that uses a strap 514 to rest the power supply 244 at the back of a patient's neck. Alternatively, the power supply 244 may be disposed on other areas of a patient's body, for example, near the waist, chest, or hip. Additionally, locations on the shoulder or arms are also available. However, in the embodiment depicted in FIG. 5A, the strap 514 is arranged for placement of the power supply 244 at the rear of the patient's neck. In order to facilitate connection and disconnection of the power supply 244 from the integrated PAP unit 1122, quick-connects or other readily removable connectors may be used, for example, connector 512 may include prongs that allow quick coupling of electrical connects 520 and 522. As discussed previously, pressurized gas from the integrated PAP unit 1122 may be used to cool the power supply 244. In the example shown in FIG. 5A, a male fitting 518 connects a cooling supply conduit 248 to the power supply 244. The male fitting 518 is configured to removably connect to the integrated PAP unit 1122. Air supplied to the power supply 244 via the cooling supply conduit 248 may escape from the power supply 244 via the cooling vents 250 along the vented cooling air path 524. Typically, in order to preserve the comfort of the patient, the air path 524 will be directed away from the patient's body, e.g., away from the neck, as shown in FIG. 5A by the arrows extending from the cooling vents 250.

As further shown in FIG. 5A, an electrical conductor 246 couples to an electrical connector 520 to distribute power from the power supply 244 to the integrated PAP unit 1122. The electrical connector 520 is disposed on a battery strap cooling and electrical male connector 516, which may combine both a mechanical connection between the strap 514 and the integrated PAP unit 1122 as well as fluid communication and electrical conductance to/from the power supply 244. Thus, relatively little stress is placed on the fluid and electrical connections.

In one embodiment, the fluid and electrical connections are omitted, and the strap 526 is provided by itself, i.e., as a purely mechanical connection.

FIG. 5B is a detailed view of a mechanical connection for the battery strap cooling and electrical male connector 516. As shown in FIG. 5B, a male nipple 530 may be used to place the power supply 244 in fluid communication with the integrated PAP unit 1122. Pressurized air from the integrated PAP unit 1122 passes through a conduit 532 disposed in a pressurized gas patch 534. In one embodiment, the pressurized gas patch 534 is formed of somewhat resilient material in order to generate a sealing effect between the pressurized gas patch 523 and the integrated PAP unit 1122. In order to latch or lock the mechanical connector in place, the battery strap cooling and electrical male connector 516 may include a connector latch 536, which locks in place as shown in FIG. 5B. Pressure on the pointed portions of the battery strap cooling and electrical male connector 516 will allow release of this connector.

FIG. 6A depicts a rotary compressor 610 and associated motor 640. The motor 640 rotates an impellor 612. Depending on the type of flow intended, different impellors 612 may be used. For example, in some applications, the veins of the impellor 612 may be swept. In other applications, the veins of the impellor 612 may be straight, i.e., directly radial. As shown in FIG. 6A, the arrows 616 demonstrate the direction of rotation of the impellor 612, and the arrows 614 indicate a flow path of the gas compressed by the impellor 612. In the embodiment shown in FIGS. 6A, the veins 618 are straight. However, as noted previously, these veins may be swept depending on the application. In any case, the compressed gas exits the rotary compressor 610 via the exhaust port 619 as pressurized exhaust gas 620.

As shown in the section view in FIG. 6B, a gas intake path 630, which is at relatively low pressure, is disposed toward a central area of the rotary compressor 610. Various levels of efficiency of the rotary compressor 610 will be achieved depending on the exhaust impellor blade width 638, intake impellor blade width 632 and shroud/impellor clearance 644. As further shown in FIG. 6B, a generally trumpet-shaped compressor shroud 636 houses the impellor 612 and includes exhaust collection duct 642, which receives the pressurized gas before discharge via the compression exhaust port 619.

FIG. 6C depicts a similar arrangement to the one shown in FIG. 6B except a gear box 650 is disposed between the motor 640 and the impellor 612. The gear box may “step up” the rotary speed of the impellor in relation to the rotary speed of the output shaft on the motor 640. In other words, the impellor will rotate at a greater rotary speed than the output shaft of the motor 640.

A gear box 650 will most frequently be used with a motor 640 when the motor 640 is a type which includes a commutator. However, a gear box 650 may be used with a motor 640, even if the motor 640 is a brushless type.

FIG. 7A depicts a gas path for inspiration-expiration from a patient. As shown in FIG. 7A, an “unassisted” breathing flow path is shown by arrows 712. This flow path is used when no pressurization is provided by the integrated PAP unit 1122.

Also shown in FIG. 7A is a compressor exhaust path 716. As shown by this arrow, gas is discharged through the membrane/heat exchanger 420 toward the patient's face, i.e., the nasal area or mouth. As noted previously, some embodiments of the integrated PAP unit 1122 may couple only with the nasal area. Other embodiments couple with the nasal area and mouth area. Further embodiments cover the eyes, nasal area, and mouth area.

The arrows 718 depict the inspiration and expiration flow path during normal use, i.e., when the integrated PAP unit 1122 is operating, and the compressor pressurizes the gas directed to the patient.

FIG. 7B depicts a section view of washout and unassisted breathing vents section. As shown in FIG. 7B, the compressor exhaust flap seat 720 abuts the diverter valve flap 310 in order to close the outlet of the compressor. This state of the diverter valve flap 310 is typical when the pressure generated by the compressor cannot overcome the elastic resistance of diverter valve flap 310, for example, when the compressor is turned off.

As further shown in FIG. 7B, the diverter valve flap 310 may be disposed in an “up” position, which provides an abutment contact between the unassisted breathing vent valve seat 722 and the diverter valve flap 310. This state of the diverter valve flap 310 is provided when the patient expires.

FIG. 8 depicts a cross-section view of an acoustic damper 314. As shown in FIG. 8, a circuitous flow path 810 winds back and forth to provide various overlapping portions. One benefit of this arrangement is that high frequency, which typically travels in generally straight lines, bounces around within the circuitous internal flow path 810 as shown by the vectors 816. In this manner, noise generated by the edges of the veins of the compressor on the intake side may be suppressed. In other words, the acoustic emissions 814 will bound around within the convoluted path 820 and be greatly diminished before exiting the integrated PAP unit 1122, for example, via the intake filter 316. In one embodiment, the internal air gas path 810 is bounded by an acoustically absorbent material 818, for example, a polymer or rubber.

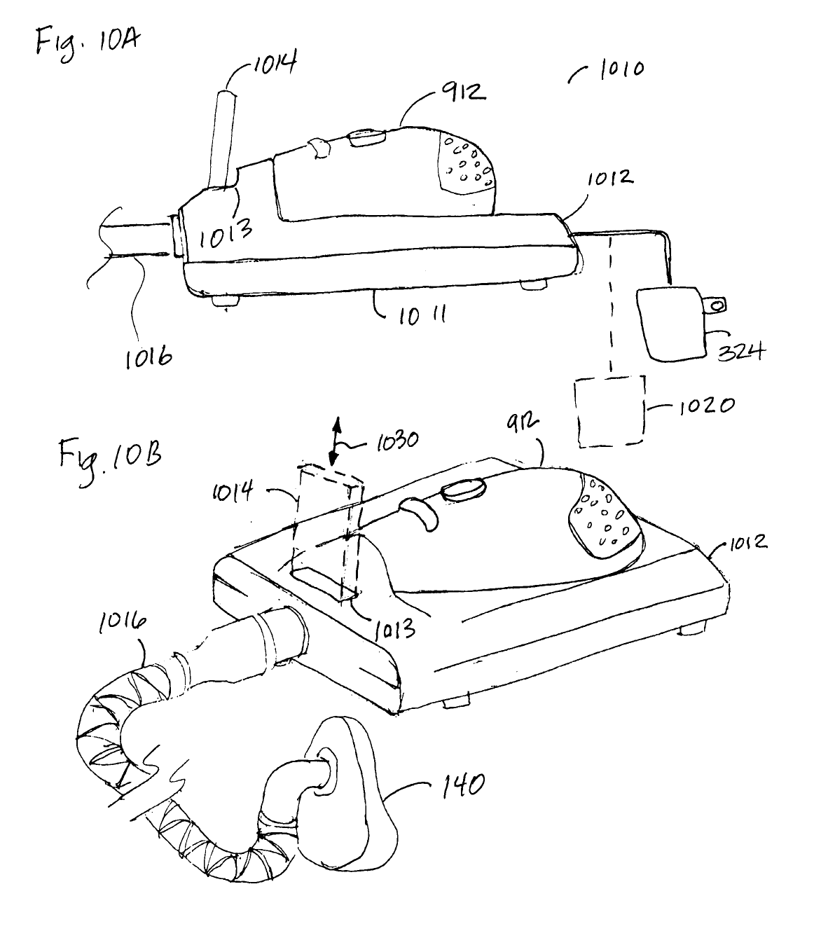

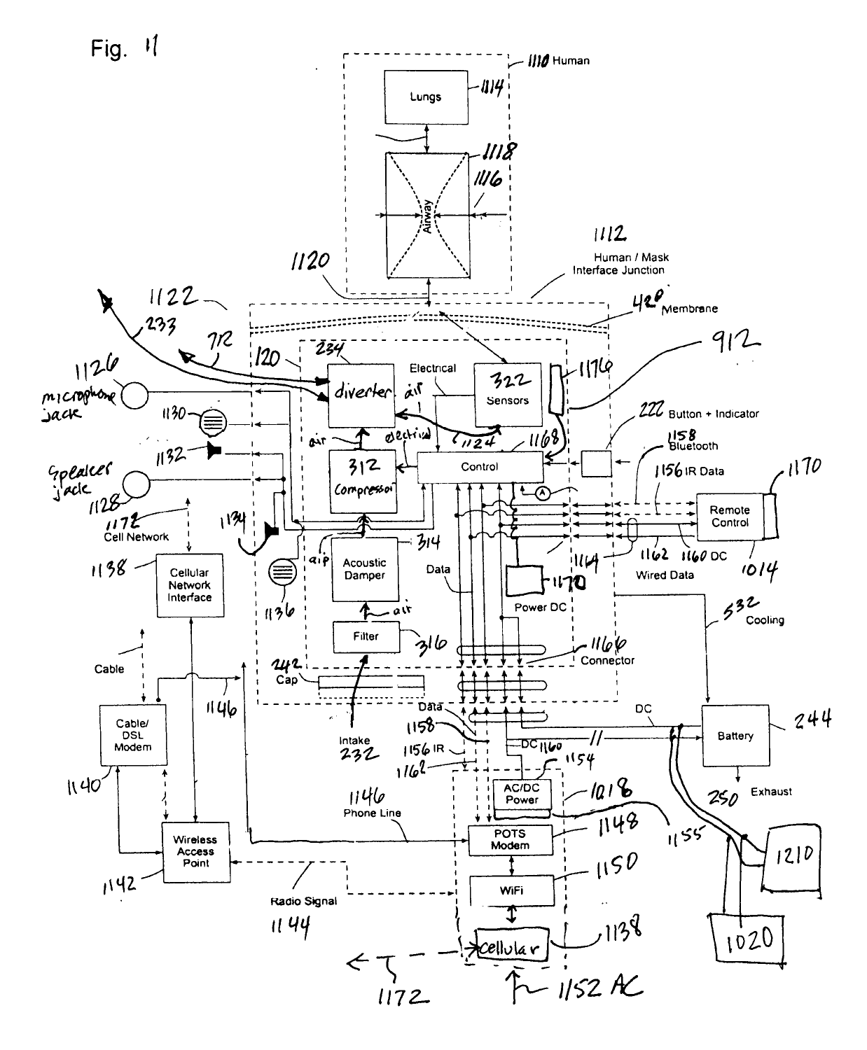

FIG. 8 depicts one example of an integrated PAP unit 1122 in which a separable flow generator 912 is couplable to a mask structure 918. In other words, the flow generator may attach and detach from the mask structure 918 via dedicated clasping devices. For example, a latch 916 may be used to secure the separable flow generator 912 to the mask structure 918. Furthermore, a hinge, preferably a separable hinge 910 may be used to further couple the separable flow generator from the mask structure 918. One benefit of the above-noted arrangement is that different flow generators 912 may be used with a given mask structure. Additionally, the separable flow generator 912 may be sent to a service center for service while the mask structure is retained by the patient. Additionally, the separable flow generator 912, in some embodiments, may be removed from the mask structure 918 and connected to a stationary base unit 1010 (shown in FIG. 10A). For example, the separable flow generator 912 may be tilted through a predetermined angle line 14 to unhinge the separable flow generator 912 from the mask structure 918. The separable flow generator 912 may then be coupled into the stationary base unit 1011 in order to supply gas to a second mask unit 140 as shown in FIG. 10B. In this way, the stationary base unit may be a relatively simple device, and all of the components included in the separable flow generator 912 may be used in conjunction either in direct contact with the mask structure or indirectly via the hose 1016 and second mask 140.

The stationary base unit may include a battery 1011 that is dedicated or rechargeable. Additionally, the stationary base unit 1010 will typically include a connection on a base 1012 for coupling to an AC adaptor 324 or automobile DC circuit adaptor 1020.

A docking receptacle 1013 typically receives the separable flow generator 912 and/or a remote control 1014, which may be charged in the docking receptacle 1013. Typically, the remote control 1014, which may be used to control the separable flow generator 912 or, in general, integrated PAP unit 1122, will be insertable and removable from the docking receptacle 1013 as shown by the arrows 1030.

FIG. 11 illustrates a systems schematic of one example of the present invention. In this example, a human 1110 interacts with the integrated PAP unit 1122 when the integrated PAP unit 1122 is affixed to the human through human mask interface junction 1112. Expiration gases and inspiration gases 1120 are transmitted through device 1122 for the purposes of assisting breathing in the treatment of sleep disorders. The gases 1120 are first transmitted bidirectionally through membrane 420 and further transmitted through sensors 322 and through flow path 1124 and 712 if diverter valve of 234 is in closed position relative to air flow from compressor 312. Gases 1120 are typically secondly transmitted bidirectionally through optional membrane/heat exchanger 420 and further transmitted through sensors 322 and through flow path 1124 and 233 if diverter valve of 234 is in an open position relative to air flow from compressor 312. Electrical signals are generated when sensors 322 are acted upon by gases 1120 and 232 whereby the electrical signals are communicated to controller 1168. The controller typically includes memory, for example, optical or magnetic memory such as RAM, ROM, or other tangible, non-transitory media, and executable software code is typically stored on the memory. Signals from sensors 322 are computed by the controller using the software, which results in controlling output of compressor 312.

Air, and optionally, other gases, are pressurized by the compressor when the intake of compressor 312 ingests environmental air 232. The environmental air 232 experiences low pressure at the intake of the integrated PAP unit 1122, and the low pressure air passes through cap 242 and penetrates the optional filter 316 whereby the air is further ingested by acoustic damper 314 and finally communicated into intake of compressor 312. Compressor 312 draws this same air (and optionally other gases, medicines, or chemicals) into its impeller where centrifugal forces act on the air from rotational energy, and resulting air is compressed and exits the compressor into diverter 234 and further through air path 1124 acting upon optional sensor(s) 322 whereby the pressurized air is further transmitted through optional membrane 420 and into airway of human 1110.

The integrated PAP unit 1122 operates when powered by power supply 244 (which may be one or more batteries) or AC adapter 1018. Battery sources include the internal battery source 1176, which is typically enclosed within flow generator, batteries disposed in power supply 244, external battery source 1210, and/or automobile DC current through automobile adapter 1020. Batteries used in any of the above-noted components may be rechargeable or non-rechargeable type. If rechargeable, the batteries can be optionally charged through electrical circuit of device 1112. As discussed previously, power supply 244 may include a pressurized gas cooling source 532.

AC adapter 1018 receives AC power 1152 and acts upon the power with AC to DC rectifier resulting in converted DC power whereby DC power is then conditioned by DC power conditioning 1155.

It is sometimes preferable to incorporate power conditioning with the controller. To achieve lightweight miniaturization of controller in flow generator 912, power conditioning is preferably located within AC adapter 1018. In still another embodiment of AC adapter 1018, a POTS modem 1148 is incorporated within the adapter which is then powered with power conditioning 1155. Further, wireless network WiFi module 1150 also powered by power conditioning 1155 can be incorporated with AC adapter 1018 or, together with POTS modem 1148 and AC adapter 1018.

The POTS modem 1148 and WiFi network module 1150 communicate with device 1122 and further with control 1168 of the device with any one of, or combination of, communication methods including infrared link 1156, wireless Bluetooth communications 1158, other wireless frequency communications, and wired electrical communications 1162. Similarly, remote control 1014 communicates with device 1122 and further with control 1168 of the device with any one of or combination of communication methods including infrared link 1156, wireless Bluetooth communications 1158, other wireless frequency communications, and wired electrical communications 1162.

Data that is logged by controller 1168 resulting from operational information and events that are recorded during treatment is typically communicated to one or more of first removable flash memory card 1170 of flow generator 120, second removable flash memory card 1170 of remote control 1014, third removable flash memory card 1170 of adapter 1018, POTS modem 1148, and WiFi module 1150, for example.

POTS modem 1148 may communicate externally through telephone line 1146 which the telephone line is further connected to telephone system. WiFi network module 1150 communicates within corresponding Wireless Network through radio signal 1144 which is then received by a wireless access point and a wireless modem 1142 which is further capable of communicating through one or more of telephone line 1146, cellular phone network 1172, and media network cable 1174. Further communication may be achieved via cellular data module 1138 of AC adapter 1018 over cellular network 1172.

Analyzing of logged data is typically performed externally to integrated PAP unit 1122 on data that is transmitted externally over one or more communication route of telephone line 1146 and then to telephone system, WiFi network signal to Internet modem and then internet network, cellular phone network 1172, and media network cable 1174.

The integrated PAP unit 1122 typically includes audio capability of one or more of an internal microphone 1136, internal speaker 1134, external microphone 1130, external speaker 1132, microphone jack 1126, and speaker jack 1128, all of which communicate with control 1168 and whereby microphone jack 1126, and speaker jack 1128 further communicate with common inputs and outputs of external audio capable devices. One or more of a microphone jack 1126 and speaker jack 1128 can also be combined into one commonly known combination jack.

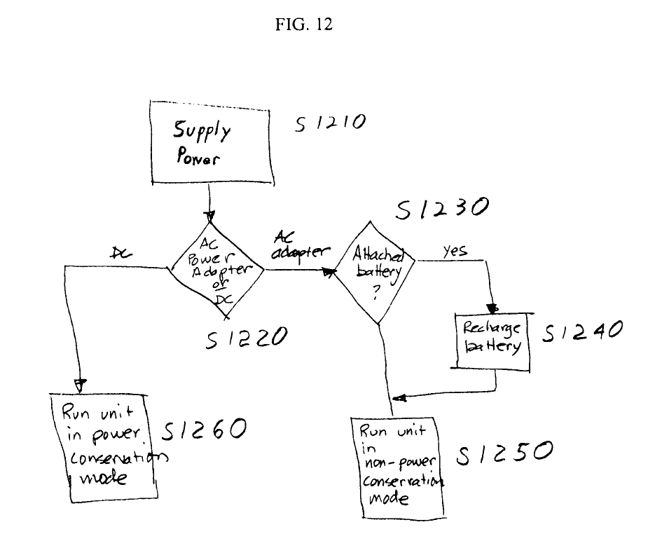

FIG. 12 depicts one example of a power management arrangement usable with the integrated PAP unit 1122. As shown in FIG. 12, when power is supplied in step S1210, a controller will determine, in step 1220, whether the power supplied is AC power or DC power. In a further embodiment, a determination is made whether, when the power is DC power, a battery is connected. This step is shown in step S1230. If a battery is determined to supply the DC power, then the battery is charged in step S1240. Additionally, a determination may be made as to whether the battery contains sufficient charge to last for a predetermined amount of time. For example, if a battery has sufficient charge for 12 hours of operation, and a patient is expected to sleep for eight hours, then the integrated PAP unit 1122 can successfully complete a sleep period for this patient. However, if the battery contains sufficient charge only for a four hour operation, and the patient intends to sleep for eight hours, it is preferable that a controller disposed within the integrated PAP unit 1122 provide a warning signal indicating that the PAP unit does not have sufficient battery power for completion of the sleep cycle. To ameliorate the above-noted problem, or, in general, when DC power is applied alone, the integrated PAP unit 1122 may run in a power conservation mode. For example, data logging, radio transmission, or other optional operations may be suspended or reduced while the integrated PAP unit 1122 is in the conversation mode depicted in step S1260. In this way, power within the battery may be conserved and the operational period available to the patient may be extended. Alternatively, when the AC adaptor is determined to supply power to the integrated PAP unit 1122, the unit may be run in a non-power conservation mode as shown in step S1250.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, define, in part, the scope of the foregoing claim terminology such that no inventive subject matter is dedicated to the public.

1-46. (canceled)

47. A gas delivery system that provides positive airway pressure therapy for a user, the system comprising:
a mask that couples to a user's face to deliver pressurized gas to an airway of the patient;
a flow generator system that pressurizes gas, the flow generator system includes at least one motor, the flow generator having an impeller and a housing defining a collection chamber for collection of the air from the impeller, an outlet from the flow generator; and
a diverter valve movable between an open and closed position and blocking the outlet from the flow generator in the closed position.

48. A gas delivery system of claim 47 wherein the flow generator is detachable from the mask.

49. A gas delivery system of claim 48 further comprising a hose interposed from the mask and the flow generator.

50. A gas delivery system of claim 48 further comprising a base unit configured to couple via a hose to the mask, wherein the flow generator system detachably directly couples to the flow generator.

51. A gas delivery system of claim 47 wherein the flow generator system is integrated with the mask, the mask having a shell defining a mask chamber and a plurality of washout vents for venting exhaust gas and further comprising an acoustic damper unit integrated with the mask and disposed upstream of the flow generator system.

52. A gas delivery system of claim 51 further comprising a moisture retention membrane within the mask chamber for the exchange of moisture into an air flow path of the mask chamber.

53. A gas delivery system of claim 51 further comprising at least one sensor in the mask chamber for monitoring of the airflow in the air flow path of the mask chamber.

54. A gas delivery system of claim 51 wherein the acoustic damper unit includes an internal pathway that repeatedly folds over upon itself to create a convoluted pathway that overlaps itself a plurality of times.

55. A gas delivery system of claim 51 further comprising:
at least one unassisted breathing orifice that allows fluid communication, separately from the flow generator system, between an exterior of the mask and an interior of the mask, the at least one unassisted breathing orifice sized to allow unencumbered/free breathing; and
the diverter valve movable from the open position that obstructs the at least one unassisted breathing orifice and allows pressurized gases from the flow generator into the interior of the mask orifice during inspiration by the patient and allows gas flow through the at least one unassisted breathing orifice during expiration by the patient and the closed position blocking the outlet from the flow generator and allowing unencumbered breathing through the at least one unassisted breathing orifice.

56. A gas delivery system of claim 47 wherein the collection chamber has a constant cross sectional area.

57. A gas delivery system of claim 47 wherein the collection chamber has an increasing cross sectional area.

58. A gas delivery system of claim 47 further comprising a controller configured to control pressure of the gas supplied by the flow generator to within a pressure range of from 0 cm/H₂O to 30 cm/H₂O.

59. A gas delivery system of claim 47 further comprising a power supply attached to the mask.

60. A gas delivery system of claim 59 wherein the power supply includes at least one battery and is connected to a strap and configured to attach to the body of the patient.

61. A gas delivery system of claim 59 wherein the power supply includes at least one battery and is connected to a strap and configured to rest against the back of the neck of the patient.

62. A gas delivery system that provides positive airway pressure therapy for a user, the system comprising:
a mask that couples to a user's face to deliver pressurized gas to an airway of the patient;
a flow generator system that pressurizes gas, the flow generator system includes at least one motor, the flow generator having an impeller and a housing defining a collection chamber for collection of the air from the impeller, an outlet from the flow generator;
the mask includes at least one washout vent that places an interior of the mask in fluid communication, separately from the flow generator system, with an exterior of the mask; and
an acoustic damper unit integrated with the mask and disposed upstream of the flow generator system.

63. A gas delivery system of claim 62 further comprising:
at least one unassisted breathing orifice that allows fluid communication, separately from the flow generator system, between an exterior of the mask and an interior of the mask, the at least one unassisted breathing orifice sized to allow unencumbered/free breathing; and
a diverter valve movable from an open position that obstructs the at least one unassisted breathing orifice and allows pressurized gases from the flow generator into the interior of the mask orifice during inspiration by the patient and allows gas flow through the at least one unassisted breathing orifice during expiration by the patient and a closed position blocking the outlet from the flow generator and allowing unencumbered breathing through the at least one unassisted breathing orifice.

64. A gas delivery system of claim 63 further comprising a moisture retention membrane within the mask chamber for the exchange of moisture into an air flow of the mask.

65. A gas delivery system of claim 63 further comprising at least one sensor in the mask chamber for monitoring of the airflow in the air flow path of the mask chamber.

66. A gas delivery system of claim 63 wherein the acoustic damper unit includes an internal pathway that repeatedly folds over upon itself to create a convoluted pathway that overlaps itself a plurality of times.

67. A gas delivery system of claim 66, wherein the acoustic damper unit comprises a resonance noise cancellation unit.

68. A gas delivery system that provides positive airway pressure therapy for a user, the system comprising:
a mask that couples to a user's face to deliver pressurized gas to an airway of the user, the mask including
a flow generator system that pressurizes gas, the flow generator system including at least one motor, the flow generator having an impeller and a housing defining a collection chamber for collection of the air from the impeller, an outlet from the flow generator;
at least one washout vent that allows constant fluid communication, separately from the flow generator system, between an exterior of the mask and an interior of the mask, the at least one washout vent sized to allow pressure to be maintained in the interior of the mask;
at least one unassisted breathing orifice that allows fluid communication, separately from the flow generator system, between an exterior of the mask and an interior of the mask, the at least one unassisted breathing orifice sized to allow unencumbered/free breathing;
a diverter valve movable from an open position that obstructs the at least one unassisted breathing orifice and allows pressurized gases from the flow generator into the interior of the mask orifice during inspiration by the patient and allows gas flow through the at least one unassisted breathing orifice during expiration by the patient and a closed position blocking the outlet from the flow generator and allowing unencumbered breathing through the at least one unassisted breathing orifice; and
an acoustic damper unit integrated with the mask and disposed upstream of the flow generator system.
